(19) United States
(12) Patent Application Publication
(10) Pub. No.: US 2016/0263189 A1
(43) Pub. Date: Sep. 15, 2016

(54) TREATMENT OF A NEURODEGENERATIVE DISEASE OR DISORDER

(71) Applicant: ACADIA Pharmaceuticals Inc., San Diego, CA (US)

(72) Inventor: Ethan S. Burstein, San Diego, CA (US)

(21) Appl. No.: 15/031,692

(22) PCT Filed: Oct. 21, 2014
(86) PCT No.: PCT/IB2014/065502

§ 371 Date: Apr. 22, 2016

Related U.S. Application Data
(60) Provisional application No. 61/894,755, filed on Oct. 23, 2013.
(60) Provisional application No. 61/902,032, filed on Nov. 8, 2013.

(51) Int. Cl. A61K 38/18; A61K 31/192

(52) U.S. Cl. CPC A61K 38/185; A61K 31/192

(57) ABSTRACT

Provided herein are methods for treating a neurodegenerative disease or disorder, or stroke using a combination of one or more RXR agonist and/or one or more Nurr1 agonist and one or more trophic factor, or pharmaceutically acceptable salts thereof. Additionally, compositions comprising of one or more RXR agonist and/or one or more Nurr1 agonist and one or more trophic factor, or pharmaceutically acceptable salts thereof for treatment of a neurodegenerative disease or disorder, or stroke are provided.

### FIELD

Provided herein are methods for treating a neurodegenerative disease or disorder, or stroke using a combination of one or more RXR agonist and/or one or more Nurr1 agonist and one or more trophic factor, or pharmaceutically acceptable salts thereof. Additionally, provided herein are compositions comprising one or more RXR agonist and/or one or more Nurr1 agonist and one or more trophic factor, or pharmaceutically acceptable salts thereof for treatment of a neurodegenerative disease or disorder, or stroke.

### BACKGROUND

Nuclear receptor related 1 protein (NURR1) also known as NR4A2 (nuclear receptor subfamily 4, group A, member 2), henceforth Nurr1 is a nuclear hormone receptor (NucHR) strongly implicated in the growth, maintenance, and survival of dopaminergic neurons, that represents a very promising therapeutic target for Parkinson's disease (PD). The essential role of Nurr1 in dopaminergic cell development was dramatically demonstrated in mouse gene knockout experiments in which homozygous mice lacking Nurr1 failed to generate midbrain dopaminergic neurons (Zetterstrom et al., 1997). Nurr1 was shown to be directly involved in the regulation of genes coding for aromatic amino acid decarboxylase, tyrosine hydroxylase (TH), and the dopamine transporter (DAT) (Hermanson et al., 2003). In addition, Nurr1 limits inflammatory responses in the central nervous system (CNS) and specifically protects dopaminergic neurons from neurotoxicity (Saijo et al., 2009). These observations suggest that Nurr1 play a pathophysiological role in aspects of neurodegenerative diseases ranging from inflammatory responses to dopaminergic nerve function and survival.

It has been shown that GDNF protects and repair dopaminergic neurons from insults such as MPTP and 6-hydroxydopamine toxicity, and axotomy (Beck et al. 1995; Bowenkamp et al. 1995; Kearns and Gash 1995; Tomac et al. 1995). Moreover, it has been demonstrated that GDNF is essential for the survival of midbrain dopamine (DA) neurons during post-natal development (Pascual et al. 2008). Because of its strong trophic actions on DA neurons, GDNF or analogs of GDNF such as neurturin are being tested clinically

RET (rearranged during transfection) is the tyrosine kinase signaling component of the receptor complex for the family ligands of the glial cell line-derived neurotrophic factor (GDNF) (Airaksinen and Saarma, 2002). Transgenic mice expressing a constitutive active mutant RET gene have increased number of midbrain DA (as assessed by tyrosine hydroxylase (TH) expression) neurons (Mijatovic et al. 2007). Conversely, mice lacking RET suffer progressive and late degeneration of dopaminergic nigro-striatal system (Kramer et al., 2007) and also show impaired capacity to regenerate dopaminergic axon terminals (Kowsky et al. 2007).

RET expression has been shown to be regulated by Nurr1 (Galleguillos et al., 2010). Specifically, Nurr1 induced the transcription of the human RET promoter in cell type and concentration-dependent manner. Conversely, knockdown of Nurr1 caused a significant reductions of both RET mRNA in the Substantia Nigra (SN) and RET protein in the striatum.

For example Nurr1 agonists have potential for treating neurodegenerative diseases such Parkinson's disease as they enhance TH and DAT expression in primary mensencephalic cultures and exert a beneficial effect on dopaminergic neurons in animal models of PD (Ordentlich et al., 2003; Jankovic et al., 2005; Dubois et al., 2006). However, the molecular basis for the actions of existing ligands is not well defined. Nurr1 may mediate its beneficial effects alone, or more likely in concert with other nuclear hormone receptor partners (Sacchetti et al., 2006; Carpentier et al., 2008). To date, there are a few examples of such ligands available for experimental testing (Shi, 2007).

Nurr1 can form dimers and is known to associate with other NucHRs including peroxisome proliferator-activated receptor gamma (PPARγ), glucocorticoid receptor (GR), farnesoid X receptor (FXR), and retinoid X receptor (RXR) (Sacchetti et al., 2006; Carpentier et al., 2008). It is currently unknown which Nurr1 interaction is therapeutically important in the treatment of PD. However, it is agreed that Nurr1 involvement in dopaminergic neuronal activation and cell survival is important (Shi, 2007). Several of the most potent Nurr1 binding compounds enhance TH and DAT expression in primary mensencephalic cultures and exert a beneficial effect on dopaminergic neurons in animal models of PD (Jankovic et al., 2005).

### SUMMARY

Provided herein are methods for treating a neurodegenerative disease or disorder, or stroke using one or more RXR agonist and/or one or more Nurr1 agonist and one or more trophic factor, or pharmaceutically acceptable salts thereof.

Also provided are compositions comprising one or more RXR agonist and/or one or more Nurr1 agonist and one or more trophic factor, or pharmaceutically acceptable salts thereof.

Also provided herein are compositions comprising one or more RXR agonist and/or one or more Nurr1 agonist and one or more trophic factor, or pharmaceutically acceptable salts thereof for treating a neurodegenerative disease or disorder, or stroke.

Also provided herein are one or more RXR agonists, such as bexarotene and one or more trophic factors such as GDNF which in combination (optionally as separate components) both administered to a subject upregulate RET.

### DETAILED DESCRIPTION OF EMBODIMENTS

### Definitions

Unless defined otherwise, all technical and scientific terms used herein have the same meaning as is commonly understood by one of ordinary skill in the art. All patents, applications, published applications and other publications referenced herein are incorporated by reference in their entirety. In the event that there is a plurality of definitions for a term herein, those in this section prevail unless stated otherwise.

The term “in combination” is intended to mean that the individual components are used in combination. This for example means that to components such as active pharmaceutical compounds are administered together, simultaneously, or a in such a manor that a combined effect is achieved. This may for example include that the components are administered separately but to give a combined effect.

The term “neurodegenerative disease or disorder” as used herein refers to a disease or disorder selected from the group consisting of Parkinson's disease, Alzheimer's disease, Huntington's disease, frontotemporal lobar degeneration associated with protein TDP-43 (FTLD-TDP, Dementia with Lewy bodies (DLB), vascular dementia, Amyotrophic lateral sclerosis (ALS), Mild Cognitive Impairment (MCI), Parkinson's disease with MCI, and other neurodegenerative related dementias due to changes in the brain caused by ageing, disease or trauma; or spinal cord injury.

The term “neuroprotection” as used herein refers to the prevention of further loss of neuronal cells, or loss of neuronal function as a result of exposure to a neurotoxin or resulting from a neurodegenerative disease or disorder. As used herein, the term “neuroprotection” is synonymous with “protection of neurons”.

As used herein, promotion of neuronal survival is considered equivalent to neuroprotection

The term “regeneration” as used herein refers to enabling an increase in the activity of an injured or disabled cell, or a cell having below normal activity relative to the natural activity of a corresponding healthy cell. Such a cell may be a neuron. In some embodiments provided herein, “regeneration” refers to the regeneration of neurons in a patient having a neurodegenerative disease or disorder.

Thus, in some embodiments “neuroregeneration” refers to the regeneration of neurons in a patient having a neurodegenerative disease or disorder. In some embodiments, “neuroregeneration refers to the process of reversing either the loss of neuronal cells, or the loss of neuronal function occurring as a result of exposure to a neurotoxin or resulting from a neurodegenerative disease.

Neurorestoration shall be defined to be equivalent to neuroregeneration.

The term “neuronal function” as used herein refers to the capability of a neuron to synthesize, store, release, transport and respond to a neurotransmitter. Thus, changes in expression or integrity of certain components of neurons, including but not limited to receptors transporters, vesicles, cell bodies, axons or dendrites may affect neuronal function.

Neurotransmitters shall be defined as diffusible molecules released by neurons that either stimulate or inhibit neuronal activity.

A “pharmaceutically acceptable salt” refers to a salt of a compound that does not abrogate the biological activity and properties of the compound. Pharmaceutical salts can be obtained by reaction of a compound disclosed herein with an acid or base. Base-formed salts include, without limitation, ammonium salt (NH₄)⁺; alkali metal, such as, without limitation, sodium or potassium, salts; alkaline earth, such as, without limitation, calcium or magnesium, salts; salts of organic bases such as, without limitation, dicyclohexylamine, piperidine, piperazine, methylpiperazine, N-methyl-D-glucamine, diethylamine, ethylenediamine, tris(hydroxymethyl)methylamine; and salts with the amino group of amino acids such as, without limitation, arginine and lysine. Useful acid-based salts include, without limitation, hydrochlorides, hydrobromides, acetates, adipates, aspartates, ascorbates, benzoates, butyrates, caparate, caproate, caprylate, camsylates, citrates, decanoates, formates, fumarates, gluconates, glutarate, glycolates, hexanoates, laurates, lactates, maleates, nitrates, oleates, oxalates, octanoates, propanoates, palmitates, phosphates, sebacates, succinates, stearates, sulfates, sulfonates, such as methanesulfonates, ethanesulfonates, p-toluenesulfonates, salicylates, tartrates, tosylates.

Pharmaceutically acceptable solvates and hydrates are complexes of a compound with one or more solvent of water molecules, or 1 to about 100, or 1 to about 10, or one to about 2, 3 or 4, solvent or water molecules.

A “prodrug” refers to a compound that may not be pharmaceutically active but that is converted into an active drug upon in vivo administration. The prodrug may be designed to alter the metabolic stability or the transport characteristics of a drug, to mask side effects or toxicity, to improve the flavor of a drug or to alter other characteristics or properties of a drug. Prodrugs are often useful because they may be easier to administer than the parent drug. They may, for example, be bioavailable by oral administration whereas the parent drug is not. The prodrug may also have better solubility than the active parent drug in pharmaceutical compositions. An example, without limitation, of a prodrug would be a compound disclosed herein, which is administered as an ester (the “prodrug”) to facilitate absorption through a cell membrane where water solubility is detrimental to mobility but which then is metabolically hydrolyzed to a carboxylic acid (the active entity) once inside the cell where water-solubility is beneficial. A further example of a prodrug might be a short peptide (polyaminoacid) bonded to an acid group where the peptide is metabolized in vivo to release the active parent compound. By virtue of knowledge of pharmacodynamic processes and drug metabolism in vivo, those skilled in the art, once a pharmaceutically active compound is known, can design prodrugs of the compound (see, e.g. Nogrady (1985) Medicinal Chemistry A Biochemical Approach, Oxford University Press, New York, pages 388-392).

“Anti-drug” refers to a compound or composition acting against or opposing illicit drugs or their use. Compounds of the present application may act as anti-drugs.

To “modulate” means the function of a bromodomain or a bromodomain containing protein means either to increase its cellular function over the base level measured in the particular environment in which it is found, or decrease its cellular function to less than the measured base level in the environment in which it is found and/or render it unable to perform its cellular function at all.

An “agonist” is defined as a compound that increases the basal activity of a receptor (i.e. signal transduction mediated by the receptor).

A “partial agonist” refers to a compound that has an affinity for a receptor but, unlike an agonist, when bound to the receptor it elicits only a fractional degree of the pharmacological response normally associated with the receptor even if a large number of receptors are occupied by the compound.

An “inverse agonist” is defined as a compound, which reduces, or suppresses the basal activity of a receptor, such that the compound is not technically an antagonist but, rather, is an agonist with negative intrinsic activity.

An “antagonist” refers to a compound that binds to a receptor to form a complex that does not give rise to any response, as if the receptor was unoccupied. An antagonist attenuates the action of an agonist on a receptor. An antagonist may bind reversibly or irreversibly, effectively eliminating the activity of the receptor permanently or at least until the antagonist is metabolized or dissociates or is otherwise removed by a physical or biological process.

A “subject” refers to an animal that is the object of treatment, observation or experiment. “Animal” includes cold- and warm-blooded vertebrates and invertebrates such as birds, fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

A “patient” refers to a subject that is being treated by a medical professional such as an M.D. or a D.V.M. to attempt to cure, or at least ameliorate the effects of, a particular disease or disorder or to prevent the disease or disorder from occurring in the first place.

A “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

A “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

An “excipient” refers to an inert substance that is added to a pharmaceutical composition to provide, without limitation, bulk, consistency, stability, binding ability, lubrication, disintegrating ability etc., to the composition. A “diluent” is a type of excipient.

A “receptor” is intended to include any molecule present inside or on the surface of a cell that may affect cellular physiology when it is inhibited or stimulated by a ligand. Typically, a receptor comprises an extracellular domain with ligand-binding properties, a transmembrane domain that anchors the receptor in the cell membrane, and a cytoplasmic domain that generates a cellular signal in response to ligand binding (“signal transduction”). A receptor also includes any intracellular molecule that in response to ligation generates a signal. A receptor also includes any molecule having the characteristic structure of a receptor, but with no identifiable ligand. In addition, a receptor includes a truncated, modified, mutated receptor, or any molecule comprising partial or all of the sequences of a receptor.

“Ligand” is intended to include any substance that interacts with a receptor.

The “Nurr1 receptor” is defined as a receptor having an activity corresponding to the activity of the Nurr1 receptor subtype characterized through molecular cloning and pharmacology. Nurr1 (nur-related factor 1, NR4A2) is an orphan nuclear hormone receptor

The “RET (rearranged during transfection) receptor” is the tyrosine kinase signaling component of the receptor complex for the glial cell line-derived neurotrophic factor (GDNF) related family of ligands including GDNF, neurturin (NRTN), artemin (ARTN), and persephin (PSPN). As used herein, “co-administration” of pharmacologically active compounds refers to the delivery of two or more separate chemical entities, whether in vitro or in vivo. Co-administration means the simultaneous delivery of separate agents; the simultaneous delivery of a mixture of agents; as well as the delivery of one agent followed by delivery of a second agent or additional agents. Agents that are co-administered are typically intended to work in conjunction with each other.

As used herein “in combination” of pharmacologically active compounds refers to the delivery of two or more separate chemical entities, whether in vitro or in vivo. In combination means the compounds may be coadministered but also that the compounds may be delivered sequentially, that is the delivery of one agent followed by delivery of a second agent or additional agents. Agents that are coadministered by sequential administration are typically intended to work in conjunction with each other. This may for example include that the components are administered separately but to give a combined effect.

The term “an effective amount” as used herein means an amount of active compound or pharmaceutical agent that elicits the biological or medicinal response in a tissue, system, animal or human that is being sought by a researcher, veterinarian, medical doctor or other clinician, which includes alleviation or palliation of the symptoms of the disease being treated.

The term “one or more” means as provided herein that it may be one or two or three or more of the specified item, for example one RXR agonist, or two Nurr 1 agonists. Thus for example two RXR agonists may be combined with one antidepressant medication.

The term “upregulation” refers to the process by which a cell increases the quantity of a cellular component, such as RNA or protein, in response to an external variable or stimulus.

The term “Parkinson's drug” refers to one or more pharmaceutically active methods or compounds to treat Parkinson's disease or symptoms caused by the disease or by other treatments not including the “Parkinson's drug”.

The “retinoid X receptor” (denoted RXR receptor) is the family of nuclear hormone receptors that are activated by 9-cis retinoic acid and not all trans retinoic acid.

RXR means one or more of RXR subtypes α, β and γ. The term “RXR agonist” refers to a compound or composition which, when combined with a Retinoid X Receptor (RXR), increases the transcriptional regulation activity of RXR homodimers and heterodimers.

The RXR agonist can include known RXR agonists that are described in, for example, the following U.S. patents and patent applications, which in their entirety are incorporated by reference herein: U.S. Pat. Nos. 5,399,586, 5,466,861, 5,780,676, and 5,801,253; U.S. patent application Ser. Nos. 07/809,980, 08/003,223, 08/027,747, 08/045,807, 08/052,050, 08/052,051, 08/179,750, 08/366,613, 08/480,127, 08/481,877, 08/872,707, and 08/944,783. See also, WO 93/11755, WO 93/21146, WO 94/15902, WO94/23068, WO 95/04036, and WO 96/20913. Other RXR agonists that can be used herein can include RXR agonists described for example, in the following articles, which in their entirety are incorporated by reference herein: Boehm et al. J. Med. Chern. 38:3146 (1994), Boehm et al. J. Med. Chern. 37:2930 (1994), Antras et al., J. Biol. Chern. 266:1157-61 (1991), Salazar-Olivo et al., Biochem. Biophys. Res. Commun. 204: 10 257-263 (1994), and Safanova, Mol. Cell. Endocrin. 104:201 (1994). Such compounds may be prepared according to methods known in the art as described in the aforementioned references, as well as in M. L. Dawson and W. H. Okamura, Chemistry and Biology of Synthetic Retinoids, Chapters 3, 8, 14 and 16, CRC Press, Inc., Florida (1990); M. L. Dawson and P. D. Hobbs, The Retinoids, Biology, Chemistry and Medicine, M. B. Sporn et al., Eds. (2nd ed.), Raven Press, New York, N.Y., pp. 5-178 (1994); Liu et al., Tetrahedron, 40:1931 (1984); Cancer Res., 43:5268 (1983); Eur. 1. Med. Chem. 15:9 (1980); Allegretto et al., J. Bio. Chem., 270:23906 (1995); Bissonette et al., Mol. Cell. Bio., 15:5576(1995); Beard et al., J. Med. Chem., 38:2820 (1995), Koch et al., J. Med. Chem., 39:3229 (1996); and U.S. Pat. Nos. 4,326,055 and 4,578,498. In some embodiments, the RXR agonists can include Bexarotene, LGD 100268, and LGD 100324. The structures of RXR agonists designated LGD 1069, LGD 100268, and LGD 100324 are shown below, and the synthesis of these compounds is described in U.S. Pat. Nos. 7,655,699 and 5,780,676. The synthesis of compounds LGD1069, LGD100268, and LGD100324 is also described in, e.g., WO 94/15902 and Boehm et al., J. Med. Chem. 38(16):3146 (1994).

More examples of RXR agonist compounds as provided herein are for example: 3,7-dimethyl-6(S),7(S)-methano,7-[1,1,4,4-tetramethyl-1,2,3,4-tetrahydronaphth-7-yl]2(E),4(E) heptadienoic acid, p[3,5,5,8,8-pentamethyl-1,2,3,4-tetrahydro-2-naphthyl-(2carbonyl)]-benzoic acid, also known as 4-[(3,5,5,8,8-pentamethyl-5,6,7,8-tetrahydro-2-naphthyl)carbonyl]benzoic acid; p(5,5,8,8-tetramethyl-1,2,3,4-tetrahydro-3-isopropyl-2-naphthyl-(2-carbonyl)]-benzoic acid, also known as 4-[(3-isopropyl-5,5,8,8-tetramethyl-5,6,7,8-tetrahydro-2-naphthyl)carbonyl]benzoic acid; p[5,5,8,8-tetramethyl-1,2,3,4-tetrahydro-3-isopropyl-2-naphthyl-(2-methano)]-benzoic acid, also known as 4-[1(3-isopropyl-5,5,8,8-tetramethyl-5,6,7,8 tetrahydro-2-naphthyl)ethenyl]benzoic acid; p[5,5,8,8-tetramethyl-1,2,3,4-tetrahydro-3-ethyl-2-naphthyl-(2-methano)]-benzoic acid, also known as 4-[1-(3-ethyl-5,5,8,8-tetramethyl-5,6,7,8-tetrahydro-2-naphthyl)ethenyl]benzoic acid; p[(5,5,8,8-tetramethyl-1,2,3,4-tetrahydro-3-bromo-2-naphthyl-(2-methano)]-benzoic acid, also known as 4-[1-(3-bromo-5,5,8,8-tetramethyl-5,6,7,8-tetrahydro-2-naphthyl)ethenyl]benzoic acid; p[5,5,8,8-tetramethyl-1,2,3,4-tetrahydro-3]-chloro-2-naphthyl-(2-methano)-benzoic acid, also known as 4-[1(3-chloro-5,5,8,8-tetramethyl-5,6,7,8-tetrahydro-2-naphthyl] ethenyl benzoic acid; p[3,5,5,8,8-pentamethyl-1,2,3,4-tetrahydro-2-naphthyl-(2-methano)]-benzoic acid, also known as 4-[1(3,5,5,8,8-pentamethyl-5,6,7,8-tetrahydro-2-naphthyl)ethenyl/benzoic acid; p[3,5,5,8,8-pentamethyl-1,2,3,4-tetrahydro-2-naphthyl-(2-hydroxymethyl)]benzoic acid, also known as 4-[1-(3,5,5,8,8-pentamethyl-5,6,7,8-tetrahydro-2-naphthyl)hydroxymethyl]benzoic acid; p[5,5,8,8-tetramethyl-1,2,3,4-tetrahydro-3-bromo-2-naphthyl-(2-carbonyl)]-benzoic acid, also known as 4-[(3-bromo-5,5,8,8-tetramethyl-5,6,7,8-tetrahydro-2-naphthyl) carbonyl]benzoic acid; p[5,5,8,8-tetramethyl-1,2,3,4-tetrahydro-3-chloro-2-naphthyl-(2-carbonyl)]-benzoic acid, also known as 4-[(3-chloro-5,5,8,8-tetramethyl-5,6,7,8-tetrahydro-2-naphthyl)carbonyl]benzoic acid; p[5,5,8,8-tetramethyl-1,2,3,4-tetrahydro-3-hydroxy-2-naphthyl-(2-carbonyl)]-benzoic acid, also known as 4-[(3-hydroxy-5,5,8,8-tetramethyl-5,6,7,8-tetrahydro-2-naphthyl)carbonyl]benzoic acid; p[5,5,8,8-tetramethyl-1,2,3,4-tetrahydro-3-ethyl-2-naphthyl-(2-carbonyl)]-benzoic acid, also known as 4-[(3-ethyl-5,5,8,8-tetramethyl-5,6,7,8-tetrahydro-2-naphthyl)carbonyl] benzoic acid; p[3,5,5,8,8-pentamethyl-1,2,3,4-tetrahydro-2-naphthyl-(2-thioketo)]-benzoic acid, also known as 4-[(3,5,5,8,8-pentamethyl-5,6,7,8-tetrahydro-2-naphthyl)thioketo] benzoic acid; p[3,5,5,8,8-pentamethyl-1,2,3,4-tetrahydro-2-naphthyl-(2-carbonyl)]-N-(4-hydroxyphenyl)benzamide, also known as 4-[(3,5,5,8,8-pentamethyl-5,6,7,8-tetrahydro-2-naphthyl)carbonyl]-N-(4-hydroxyphenyl)benzamide; p[3,5,5,8,8-pentamethyl-1,2,3,4-tetrahydro-2-naphthyl-(2-methano)]-N-(4-hydroxyphenyl)benzamide, also known as 4-[1-(3,5,5,8,8-pentamethyl-5,6,7,8-tetrahydro-2-naphthyl)ethenyl]-N-(4-hydroxyphenyl)benzamide; 2-[1-(3,5,5,8,8-pentamethyl-5,6,7,8-tetrahydro-2-naphthyl)ethenyl]pyridine-5-carboxylic acid; ethyl 2-[1-(3,5,5,8,8-pentamethyl-5,6,7,8-tetrahydro-2-naphthyl)ethenyl]pyridine-5-carboxylate; 2-[1-(5,5,8,8-tetramethyl-5,6,7,8-tetrahydro-2-naphthyl)ethenyl]pyridine-5-carboxylic acid; 4-[1-(3,5,5,8,8-pentamethyl-5,6,7,8-tetrahydro-2-naphthyl)epoxy]benzoic acid; 4-[1-(3,5,5,8,8-pentamethyl-5,6,7,8-tetrahydro-2-naphthyl)cyclopropyl]benzoic acid; 4-[1-(3,5,5,8,8-pentamethyl-5,6,7,8-tetrahydro-2-naphthyl)ethenyl]benzenetetrazole; 5-[1-(3,5,5,8,8-pentamethyl-5,6,7,8-tetrahydro-2-naphthyl)ethenyl]pyridine-2-carboxylic acid; 2-[1-(3,5,5,8,8-pentamethyl-5,6,7,8-tetrahydro-2-naphthyl)cyclopropyl]pyridine-5-carboxylic acid; methyl 2-[1-(3,5,5,8,8-pentamethyl-5,6,7,8-tetrahydro-2-naphthyl)cyclopropyl]pyridine-5-carboxylate; 3-methyl-7-propyl-9-(2,6,6-trimethyl-1-cyclohexen-1-yl)-2E,4E,6Z,8E nonatetranoic acid; 3-methyl-7-isopropyl-9-(2,6,6-trimethyl-1-cyclohexen-yl)-2E,4E,6Z,8E nonatetranoic acid; 3-methyl-7-t-butyl-9-(2,6,6-trimethyl-1-cyclohexen-1-yl)-2E,4E,6Z,8E nonatetranoic acid; 3-methyl-5-{2-12-(2,6,6-trimethylcyclohexen-1-yl)ethenyl-1-cyclohexyl}-2E,4E-pentadienoic acid; (2E,4E)-3-methyl-5-[1-(3,5,5,8,8-pentamethyl-5,6,7,8-tetrahydro-2-naphthyl)cyclopropyl]penta-2,4-dienoic acid; (2E,4E)-3-methyl-6-(1-[2,6,6-trimethyl-1-cyclohexenyl)ethenyl]cyclopropyl)-2,4-hexadienoic acid; (2E,4E,6Z)-7-(5,5,8,8-tetramethyl-5,6,7,8-tetrahydro-2-naphthyl)-3,8-dimethyl-nona-2,4,6-trienoic acid; (2E,4E,6Z)-7-(3,5,5,8,8-pentamethyl-5,6,7,8-tetrahydro-2-naphthyl)-3-methylocta-2,4,6-trienoic acid; 2-[1-(5,5,8,8-tetramethyl-5,6,7,8-tetrahydro-2-naphthyl)cyclopropyl] pyridine-5-carboxylic acid; 4-[(3,5,5,8,8-pentamethyl-5,6,7,8-tetrahydro-2-naphthyl)carbonyl] benzoic acid oxime; 4-[(3,5,5,8,8-pentamethyl-5,6,7,8-tetrahydro-2-naphthyl)carbonyl] benzoic acid methyloxime; 4-[1-(2-methyl-4-t-butylphenyl)ethenyl] benzoic acid; 4-[1-(2-methyl-4-t-butylphenyl)cyclopropyl] benzoic acid; 4-[(2-methyl-4-t-butylphenyl)carbonyl] benzoic acid; 4-[(2-methyl-4-t-butylphenyl)carbonyl] benzoic acid oxime; and 4-[1-(2-methyl-4-t-butylphenyl)carbonyl] benzoic acid methyloxime; or a pharmaceutically acceptable salt thereof

A Nurr1 agonist as provided herein, is for example, selected from one or more of the following:

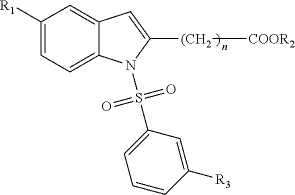

| Ex. | R₁ | n | R₂ | R₃ |
|---|---|---|---|---|
| 1 | 5-CF₃ | 3 | CH₃ | CH(CH₃)₂ |
| 2 | 5-CF₃ | 3 | H | CH(CH₃)₂ |
| 3 | 5-Cl | 3 | CH₃ | CH(CH₃)₂ |
| 4 | 5-Cl | 3 | H | CH(CH₃)₂ |
| 5 | 5-CF₃ | 4 | C(CH₃)₃ | CH(CH₃)₂ |
| 6 | 5-CF₃ | 4 | H | CH(CH₃)₂ |
| 7 | 5-CF₃ | 3 | CH₃ | C(CH₃)₃ |
| 8 | 5-CF₃ | 3 | H | C(CH₃)₃ |
| 9 | 5-CF₃ | 4 | CH₃ | C(CH₃)₃ |
| 10 | 5-CF₃ | 4 | H | C(CH₃)₃ |

or (6-Chloroimidazo[1,2-a]pyridin-2-yl)(pyridin-2-yl) methanone; Benzoxazol-2-yl(6-chloroimidazo[1,2-a]pyridin-2-yl)methanone; (6-Chloroimidazo[1,2-a]pyridin-2-yl)(3-fiJryl)methanone; (6-Chloroimidazo[1,2-a]pyridin-2-yl)(thien-2-yl)methanone; (6-Chloroimidazo[1,2-a]pyridin-2-yl)(thien-3-yl)methanone; 1,3-Benzodioxol-5-yl(6-chloroimidazo[1,2-a]pyridin-2-yl)methanone; Benzothiazol-2-yl(6-chloroimidazo[1,2-a]pyridin-2-yl) methanone; (6-Methylimidazo[1,2-a]pyridin-2-yl)(thien-2-yl)methanone; (5-Methylimidazo[1,2-a]pyridin-2-yl)(thien-2-yl)methanone, (6-Pyridin-2-yl)imidazo[1,2-a]pyridin-2-yl)(thien-2-yl) methanone, or pharmaceutically acceptable salts thereof.

Additional examples of Nurr1 agonist are found and indicated in the following table:

| Name | Structure | Reported Pharmacology | Reference |
|---|---|---|---|
| 9-cis-Retinoic Acid | 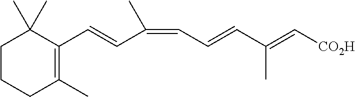 [SMILES: CC1=C(/C=C/C(C)=C\C=C\C(C)=C\C(=O)O)C(C)(C)CCC1] | Non-selective full retinoid agonist | |
| Bexarotene (Targretin/LGD1069) | 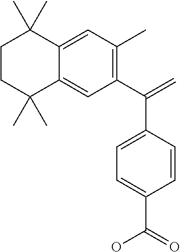 [SMILES: C=C(c1ccc(C(=O)O)cc1)c1cc2c(cc1C)C(C)(C)CCC2(C)C] | RXR selective agonist | Boehm et al., J. Med. Chem., 1994. |
| LG 100268 | 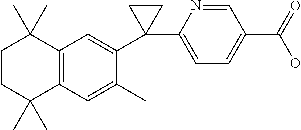 [SMILES: Cc1cc2c(cc1C1(c3ccc(C(=O)O)cn3)CC1)C(C)(C)CCC2(C)C] | RXR selective agonist | Boehm et al., J. Med. Chem., 1995. |
| SR11237 | 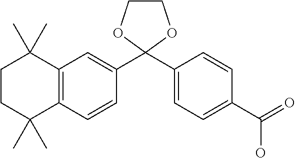 [SMILES: CC1(C)CCC(C)(C)c2cc(C3(c4ccc(C(=O)O)cc4)OCCO3)ccc21] | RXR selective agonist | Wallen-Mackenzie et al., Genes Dev., 2003. |
| XCT0135908 | 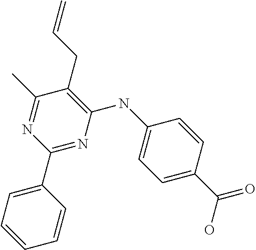 [SMILES: C=CCc1c(C)nc(-c2ccccc2)nc1Nc1ccc(C(=O)O)cc1] | RXR-Nurr1 heterodimer selective agonist | Wallen-Mackenzie et al., Genes Dev., 2003. |
| HX630 | 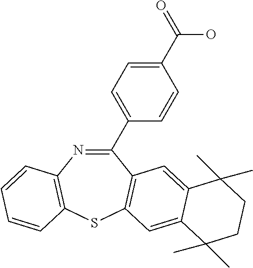 [SMILES: CC1(C)CCC(C)(C)c2cc3c(cc21)Sc1ccccc1N=C3c1ccc(C(=O)O)cc1] | RXR selective agonist/potentiator | compound 29 in Umemiya et al., J. Med. Chem., 1997. |
| PAO24 | 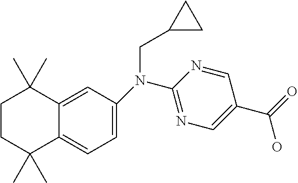 [SMILES: CC1(C)CCC(C)(C)c2cc(N(CC3CC3)c3ncc(C(=O)O)cn3)ccc21] | RXR selective agonist/potentiator | compound 10G in Ohta et al., J. Med. Chem., 2000. |
| AC-261066 | 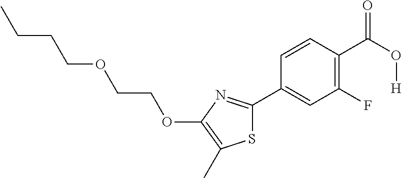 [SMILES: CCCCOCCOc1nc(-c2ccc(C(=O)O)c(F)c2)sc1C] | RARb2 selective agonist | Lund et al., J. Med. Chem., 2005. |
| Honokiol | 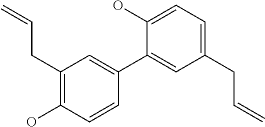 [SMILES: C=CCc1ccc(O)c(-c2ccc(O)c(CC=C)c2)c1] | RXR selective agonist/potentiator | Kotani et al., J. Nat Prod., 2010. |
| AC271251 | 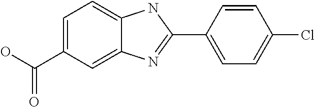 [SMILES: O=C(O)c1ccc2[nH]c(-c3ccc(Cl)cc3)nc2c1] | Putative Nurr1 agonist | compound 11 in Dubois et al., Chem. Med. Chem., 2006. |
| AC271252 | 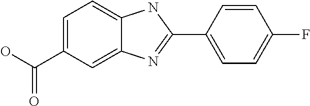 [SMILES: O=C(O)c1ccc2[nH]c(-c3ccc(F)cc3)nc2c1] | Putative Nurr1 agonist | compound 12 in Dubois et al., Chem. Med. Chem., 2006. |
| 6-MP | 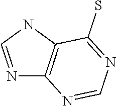 | Putative Nurr1 agonist/ anticancer drug | Ordentlich et al., J. Biol. Chem., 2003. |
| 6-MP 2-deoxyribose | 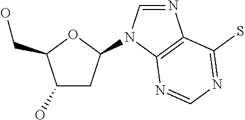 [SMILES: OC[C@H]1O[C@@H](n2cnc3c(S)ncnc32)C[C@@H]1O] | 6-MP active metabolite | Ordentlich et al., J. Biol. Chem., 2003. |
| 6-MP ribose | 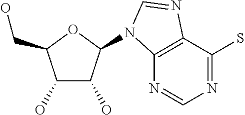 [SMILES: OC[C@H]1O[C@@H](n2cnc3c(S)ncnc32)[C@H](O)[C@@H]1O] | 6-MP active metabolite | Ordentlich et al., J. Biol. Chem., 2003. |

There is a need for compounds, such as Nurr1 agonists, or compounds that induce activation of Nurr1 indirectly through Nurr1 binding partners that are neuroprotective via activity at the Nurr1 receptor in the central nervous system, both as pharmacological tools and as therapeutic agents. Because such compounds may increase expression of RET, using such compounds in combination with GDNF, or analogs of GDNF which utilize RET to exert trophic effects on DA neurons would be particularly beneficial.

Combining bexarotene, or another RXR or Nurr1 agonist with one of the trophic factors listed herein may offer superior efficacy as neuroprotective agents than any of these agents alone, while maintaining an acceptable or improved side effect and safety profile. For example, bexarotene may have the potential to ‘prime’ neurons, rendering them more responsive to the trophic, neurogenic, and neuroprotective actions of other agents, such as those described above, by driving transcriptional upregulation of receptors such as RET.

For example, combining RXR agonists, or Nurr1 agonists, with trophic factors may produce greater neuroprotective activity together than the sum of the neuroprotective activity afforded by each agent separately. In some embodiments, sub-effective concentrations (concentrations that do not provide significant neuroprotection administered alone) of bexarotene are combined with sub-effective concentrations of GDNF to provide greater neuroprotective activity against the neurotoxin MPP+ than either agent alone and greater than the sum of neuroprotective activity of each agent given alone (i.e. a synergistic effect is obtained), for example, as shown in FIG. 3 and FIG. 4. In some embodiments, sub-effective concentrations of bexarotene are combined with sub-effective concentrations of GDNF to provide greater neuroprotective activity against the neurotoxic peptide alpha-synuclein than either agent alone, or than the sum of neuroprotective activity of each agent given alone, for example, as shown in FIG. 5. In some embodiments, effective concentrations of bexarotene are combined with effective concentrations of GDNF to promote greater neuronal survival than neurons not challenged with any toxin, as shown in FIG. 6. The results provided herein allow those skilled in the art to appreciate the potential benefits of combining one or more a RXR agonists, or Nurr1 agonists, with one or more trophic factors.

Also provided herein is a method for upregulation of RET, for example when a trophic factor such as GDNF and bexarotene are combined.

Also provided herein is a method for treating a neurodegenerative disease or disorder, or stroke using one or more RXR agonist and/or one or more Nurr1 agonist and one or more trophic factor, or pharmaceutically acceptable salts thereof.

Also provided herein is a composition comprising one or more RXR agonist and/or one or more Nurr1 agonist and one or more trophic factor, or pharmaceutically acceptable salts thereof.

Also provided herein is a composition comprising one or more RXR agonist and/or one or more Nurr1 agonist and one or more trophic factor, or pharmaceutically acceptable salts thereof for treating a neurodegenerative disease or disorder, or stroke.

In some embodiments the neurodegenerative disease relates to disease or disorders selected from the group consisting of Parkinson's disease, Alzheimer's disease, Huntington's disease, frontotemporal lobar degeneration associated with protein TDP-43 (FTLD-TDP, Dementia with Lewy bodies (DLB), vascular de-mentia, Amyotrophic lateral sclerosis (ALS), Mild Cognitive Impairment (MCI), Parkinson's disease with MCI, and other neurodegenerative related dementias due to changes in the brain caused by ageing, disease or trauma; or spinal cord injury. In some embodiments the RXR agonist is selected from Bexarotene and 3,7-dimethyl-6(S),7(S)methano,7-[1,1,4,4-tetramethyl-1,2,3,4-tetrahydronaphth-7-yl]2(E),4(E) heptadienoic acid. In some embodiments the trophic factor is selected from the group consisting of glial-cell-line-derived neurotrophic factor (GDNF) or analogs of GDNF, Neurturin (NTN), brain-derived neurotrophic factor (BDNF), fibroblast growth factor (FGF), fibroblast growth factor 9 (FGF-9), ciliary neurotrophic factor (CNTF), bone morphogenetic proteins (BMPs), mesencephalic astrocyte-derived neurotrophic factor (MANF), and Cerebral dopamine neurotrophic factor also called Conserved dopamine neurotrophic factor (CDNF), hepatocyte growth factor (HGF), nerve growth factor (NGF), Neurotrophin 3 (NT-3), Neurotrophin 4/5 (NT-4/5), Neurotrophin 6 (NT-6), Neurotrophin 7 (NT-7), artemin (ARTN), and persephin (PSPN), CERE-120 (AAV2 vector encoding human neurturin), granulocyte macrophage colony-stimulating factor (GM-CSF), Insulin-like growth factor (IGF)-1, transforming growth factor beta 1 (TGF-beta1), In some embodiment the trophic factor is GDNF, BDNF, NGF or NTN. In some embodiments the trophic factor is administered by means of a pump connected to a brain-implantable catheter.

In some embodiments GDNF is administered by means of a pump connected to a brain-implantable catheter.

In some embodiments the trophic factor is administered by injection of a vector expressing the gene encoding the trophic factor, such as an adeno-associated viral (AAV) vector, such as wherein the AAV vector is serotype 2 (AAV2).

A vector is a gene therapy delivery vehicle, or carrier, that encapsulates therapeutic genes for delivery to cells. These include both genetically disabled viruses such as adenovirus and nonviral vectors.

Adeno-associated virus (AAV) mean a viral vector system for gene therapy delivery including a small virus from the parvovirus family which is a small virus with a genome of single stranded DNA which infects human cells and can insert genetic material into the human genome, and a gene encoding a therapeutic agent and AAV2 shall mean an AAV vector with serotype 2.

Additionally “vg” shall mean viral genomes, a means of calculating the dosage of an AAV vector delivered gene encoding a therapeutic agent.

CERE-120 shall mean an AAV2 vector carrying a gene encoding NRTN, CERE-110 shall mean an AAV2 vector carrying a gene encoding NGF, CERE-130 or CERE-135 shall mean an AAV2 vector carrying a gene encoding IGF-1, CERE-140 shall mean an AAV2 vector carrying a gene encoding NT4.

In some embodiments the vector is selected from one or more from the group consisting of CERE-120, CERE-110, CERE-130, CERE-135, CERE-140.

In some embodiments the vector is CERE-120 and/or CERE-110.

In some embodiments CERE-120 and/or CERE-110 is administered by intracerebral injection, for example CERE-120 can be injected into the substantia nigra (SN), and/or into the putamen, and CERE-110 is injected into the basal forebrain region of the brain containing the nucleus basalis of Meynert (NBM). Examples of doses are: CERE-120 administered in a total ranging from 1×10¹⁰ vg to 1×10¹³ vg, such as 1×10¹¹ vg to 6×10¹² vg, such as 1×10¹¹ vg to 3×10¹² vg. Examples thereof are about 1.3×10¹¹ vg, about 4×10¹¹ vg, such as about 5.4×10¹¹ vg, such as about 1×10¹² vg. Another example is about 2×10¹² vg to the putamen, and 4×10¹¹ vg to the substantia nigra.

In some embodiments CERE-110 administered in a total dose ranging from 5×10⁹ vg to 5×10¹² vg, such as 5×10⁹ vg to 1×10¹² vg, for example 2×10¹⁰ vg, such as about 1.0×10¹¹ vg or such as about 2.0×10¹¹ vg.

In some embodiments, CERE-110 is administered by 2-7, such as 3-5, such as 4 stereotactic injections targeted to the NBM. In some embodiments two sites of the NBM are targeted.

In some embodiments CERE-120 is administered by 2-7, such as 3-5, such as 4stereotactic injections per hemisphere into the putamen.

In some embodiments CERE-120 is administered by 2-7, such as 3-5, such as 3stereotactic injections per hemisphere into the putamen

In some embodiments CERE-120 is administered by 1-4, such as 1-2, such as 1 injection(s) per hemisphere into the substantia nigra (SN).

In some embodiments CERE-120 is administered by a combination of 2-7, such as 3-5, such as 3 injections per hemisphere into the putamen and 1-4, such as 1-2, such as 1 injection(s) per hemisphere into the substantia nigra (SN).

In some embodiments CERE-120 is administered by a combination of 3 injections per hemisphere into the putamen and 1 injection per hemisphere into the substantia nigra (SN).

In some embodiments the tracts for stereotactic injections of CERE-120 are separated by about 5 mm.

In some embodiments two or more deposits per injection are made along the same tract for stereotactic injections of CERE-120

In some embodiments one of these deposits is ventral and one is rostral, separated by about 4 mm.

In some embodiments the infusion rate for stereotactic injections of CERE-110 or CERE-120 is about 1 to about 4 μl/min, such as 2 to about 3 μl/min.

In some embodiments the RXR agonist is bexarotene. Bexarotene can for example be administered in a dose of at least 0.05 mg/day, such as 0.05-600 mg/day, such as 0.05-300 mg/day, such as 0.05-150 mg/day, such as 0.05-75 mg/day or 75-150 mg/day. In some embodiments the trophic factor is selected from the group consisting of NRTN, GDNF, IGF-1, NGF, or NT4 and the RXR agonist is bexarotene.

In some embodiments the trophic factor is GDNF and the RXR agonist is bexarotene. In some embodiments, provided herein is a method for treating a neurodegenerative disease or disorder, or stroke using a RXR agonist and/or a Nurr1 agonist and a trophic factor

In some embodiments the trophic factor and the RXR agonist and/or Nurr1 agonist are administered to a subject. In some embodiments the RXR agonist is bexarotene and the trophic factor is GDNF, which in some embodiments upregulate RET.

### DESCRIPTION OF THE DRAWINGS

In the following examples reference is made to the appended drawings which illustrate the following.

FIG. 1 shows the effect of 1 mg/kg/day of bexarotene administered subcutaneously normalized Ret immunolabeling in the SNc. Sprague-Dawley rats received saline infusions (denoted sham) or bilateral infusions of 6OHDA into the SNc (denoted lesion) as described (McFarland et al., 2013). Infusion of 6OHDA caused a reduction in the number of Ret positive cells compared to sham animals. Bexarotene treatment starting 3 days after 6OHDA infusion for 28 days reversed the loss of RET positive cells, and increased RET expression above the levels in the sham animals. A), RET positive cells expressed as % of sham; B), sham; C), vehicle treated lesioned animals; D) bexarotene treated lesioned animals. Shown are representative images of substantia nigral tissue immunolabeled for Ret. Subcutaneous treatment with bexarotene (16 mM or 1 mg/kg/day based on the volume delivered per day and the starting weights of the rats) for 28 days beginning 3 days after lesion resulted in significantly improved Ret immunolabeling. *indicates a significant difference from sham controls, p<0.05; and + indicates a significant difference from Lesion/Veh animals, p<0.05.

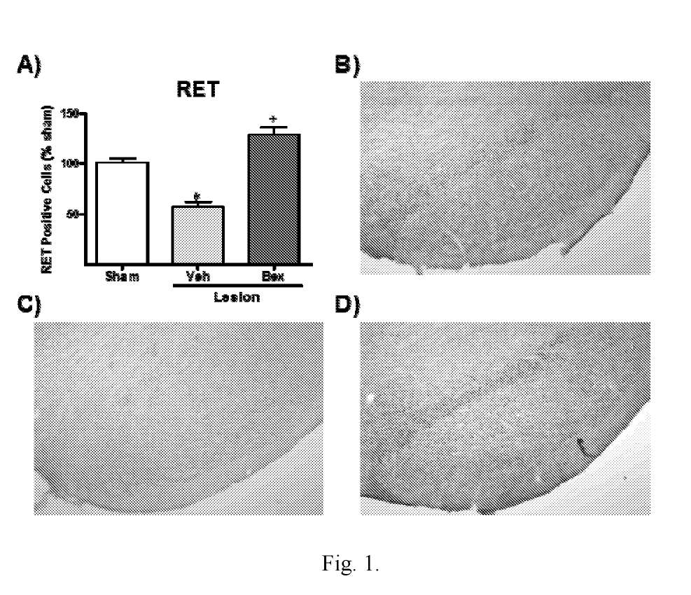

FIG. 2 shows the effect of 1 and 3 mg/kg/day of bexarotene orally administered for 4 days to rats that received unilateral striatal injections of 6-OHDA 24 hrs prior to receiving drug treatment. This toxin treatment protocol reduces expression of RET without reducing DA cell number in the SNc. Six hours after the fourth dose, the animals (n=5 per group) were sacrificed, brains were rapidly removed, and the ventral midbrain was dissected and snap-frozen. mRNA was isolated using the RiNeasy Mini kit (Qiagen) according to the supplier's recommendations. RNA concentration was determined using the NanoDrop (Thermo Scientific) and a 500 ng quantity of RNA was used for the reverse transcription performed with random primers (Invitrogen) and SuperScriptIII (Invitrogen) according to the manufacturer's recommendations. Primers were designed using Primer Blast (NIH, USA). SYBR® green quantitative real-time PCR was performed with LightCycler 480 SYBR® Green I Master (Roche) using standard procedures. Data were quantified using the ΔΔCt-method and normalized to GAPDH (Glyceraldehyde 3-phosphate dehydrogenase) and β-actin expression. Data are reported as fold change.

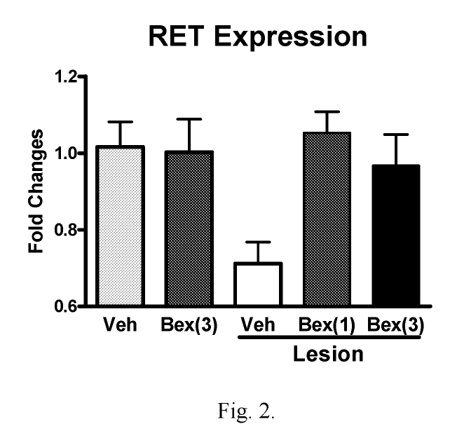

FIG. 3 illustrates the synergistic effects of sub-effective doses (doses that do not provide significant neuroprotection administered alone) of bexarotene and sub-effective doses of GDNF applied to primary cultures of dopaminergic neurons previously exposed to MPP+. Specifically, rat dopaminergic neurons derived from fetal (15 day gestation) midbrains were cultured as described (Schinelli et al., 1988). On day 6 of culture, medium was removed and fresh medium added, without or with 4 μM MPP+. On day 7, the culture was washed with fresh medium without (containing vehicle) or with test drugs for 48 h. After 48 h, cells were fixed (all conditions) by paraformaldehyde 4% solution, permeabilized with 0.1% saponin (Sigma), and labeled for TH as described (McFarland et al., 2013). Shown are the effects of bexarotene and GDNF at the indicated concentrations, given either separately or together on TH positive neurons after a 24 h MPP+ injury (4 μM) expressed as percent of control cells not treated with MPP+. Specifically, 0.3, 1 or 3 nM concentrations of bexarotene alone were not able to significantly increase the % of TH positive cells compared to cells treated with MPP+ alone. Similarly, 0.31, 0.62 or 1.25 ng/ml concentrations of GDNF alone were not able to significantly increase the % of TH positive cells compared to cells treated with MPP+ alone. However, when 3 nM bexarotene was administered together with 0.31, 0.62 or 1.25 ng/ml concentrations of GDNF, the combination of treatments was able to significantly increase the % of TH positive cells compared to cells treated with MPP+ alone. Similarly, when 1 nM bexarotene was administered together with 1.25 ng/ml concentrations of GDNF, the combination of treatments was able to significantly increase the % of TH positive cells compared to cells treated with MPP+ alone. These results show that combinations of bexarotene and GDNF are able to restore the TH phenotype of DA neurons under conditions where neither can alone.

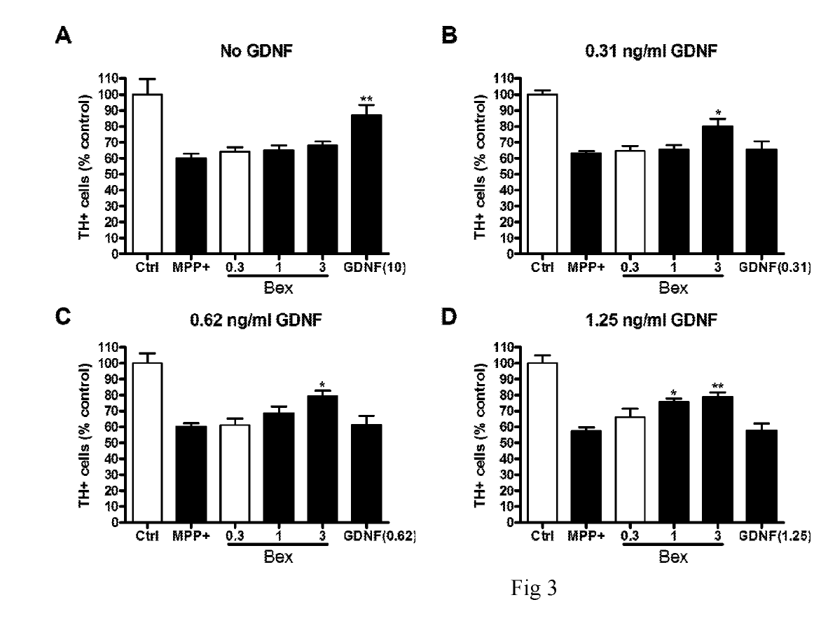

FIG. 4 illustrates the synergistic effects of sub-effective doses of bexarotene and GDNF applied to primary cultures of dopaminergic neurons previously exposed to MPP+. Cultured neurons were treated as described above and treated with 2 ng/ml GDNF, 5 nM bexarotene, or the combination. GDNF alone caused a 3 percent increase in TH positive neurons over the MPP alone condition. Bexarotene alone caused a 9 percent increase in TH positive neurons over the MPP alone condition. GDNF combined with bexarotene caused a 25 percent increase in TH positive neurons over the MPP alone condition.

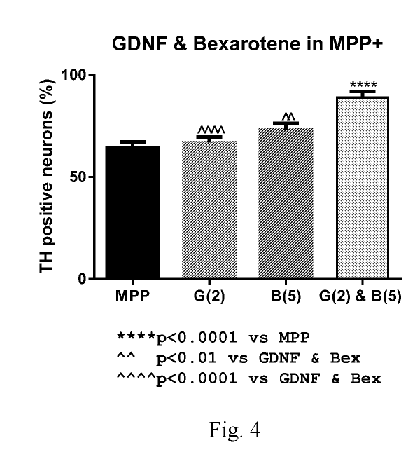

FIG. 5 shows the synergistic effects sub-effective doses of bexarotene and GDNF applied to primary cultures of dopaminergic neurons previously exposed to alpha-synuclein (α-syn). Cultured neurons were treated as described above. On day 7, the media was removed, and fresh media with the α-synuclein peptide (250 nM) was added. On day 8, the media was removed, and fresh media with the α-synuclein peptide (250 nM) and the indicated drug treatments was added. After 96 h, cells were fixed (all conditions) by paraformaldehyde 4% solution, permeabilized with 0.1% saponin (Sigma), and labeled for TH. GDNF alone (12.5 ng/ml) caused an 8 percent increase in TH positive neurons over the α-synuclein alone condition. Bexarotene alone (10 nM) caused an 8 percent increase in TH positive neurons over the α-synuclein alone condition. GDNF combined with bexarotene caused a 25 percent increase in TH positive neurons over the α-synuclein alone condition.

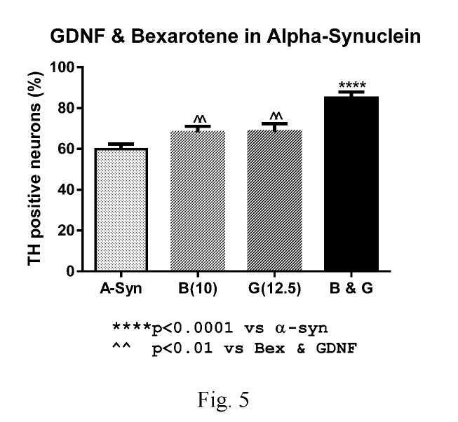

FIG. 6 shows that effective doses of bexarotene combined with effective doses of GDNF promote greater neuronal survival than the control (sham or untreated) neurons. Cultured neurons were treated with alpha-synuclein as described above. GDNF alone (100 ng/ml) caused 96 percent survival of TH positive neurons compared to the control condition. Bexarotene alone (100 nM) caused 88 percent survival of TH positive neurons compared to the control condition. GDNF combined with bexarotene caused 110 percent survival of TH positive neurons compared to the control condition.

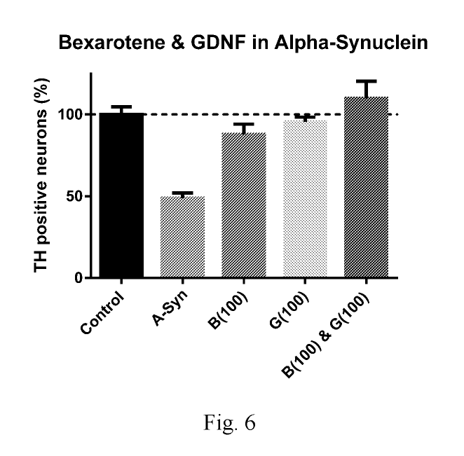

### REFERENCES (WHICH IN THEIR ENTIRETY ARE INCORPORATED HEREIN)

Airaksinen M S, Saarma M. The GDNF family: signaling, biological functions and therapeutic value. Nat Rev Neurosci. 2002 May; 3(5):383-94.
Boehm M F, Zhang L, Badea B A, White S K, Mais D E, Berger E, Suto C M, Goldman M E, Heyman R A. (1994) Synthesis and structure-activity relationships of novel retinoid X receptor-selective retinoids. J Med Chem 37, 2930-2941.
Boehm M F, Zhang L, Zhi L, McClurg M R, Berger E, Wagoner M, Mais D E, Suto C M, Davies J A, Heyman R A, Nazdan A M. (1995) Design and synthesis of potent retinoid X receptor selective ligands that induce apoptosis in leukemia cells. J Med Chem 38, 3146-3155.
Beck K. D., Valverde J., Alexi T., Poulsen K., Moffat B., Vandlen R. A., Rosenthal A. and Hefti F. (1995) Mesencephalic dopaminergic neurons protected by GDNF from axotomy-induced degeneration in the adult brain. Nature 373, 339-341.
Bowenkamp K. E., Hoffman A. F., Gerhardt G. A., Henry M. A., Biddle P. T., Hoffer B. J. and Granholm A. C. (1995) Glial cell line derived neurotrophic factor supports survival of injured midbrain dopaminergic neurons. J. Comp. Neurol. 355, 479-489.
Carpentier, R., P. Sacchetti, et al. (2008). “The glucocorticoid receptor is a co-regulator of the orphan nuclear receptor Nurr1.” J Neurochem 104(3): 777-89.
Dubois, C., B. Hengerer, et al. (2006). “Identification of a potent agonist of the or-phan nuclear receptor Nurr1.” Chem Med Chem 1(9): 955-8.
Galleguillos D, Fuentealba J A, Gomez L M, Saver M, Gomez A, Nash K, Burger C, Gysling K, Andres M E. Nurr1 regulates RET expression in dopamine neurons of adult rat midbrain. J Neurochem. 2010 August; 114(4):1158-67
Hermanson, E., B. Joseph, et al. (2003). “Nurr1 regulates dopamine synthesis and storage in MN9D dopamine cells.” Exp Cell Res 288(2): 324-34.
Kearns C. M. and Gash D. M. (1995) GDNF protects nigral dopamine neurons against 6-hydroxydopamine in vivo. Brain Res. 672, 104-111.
Kramer E. R., Aron L., Ramakers G. M., Seitz S., Zhuang X., Beyer K., Smidt M. P. and Klein R. (2007) Absence of Ret signaling in mice causes progressive and late degeneration of the nigrostriatal system. PLoS Biol. 5, e39.
Kotani H, Tanabe H, Mizukami H, Makishima M, Inoue M. (2010) Identification of a naturally occurring rexinoid, honokiol that activates the retinoid X receptor. J Nat Prod 73, 1332-1336.
Kowsky S., Poppelmeyer C., Kramer E. R., Falkenburger B. H., Kruse A., Klein R. and Schulz J. B. (2007) RET signaling does not modulate MPTP toxicity but is required for regeneration of dopaminergic axon terminals. Proc. Natl Acad. Sci. USA 104, 20049-20054.
Jankovic, J., S. Chen, et al. (2005). “The role of Nurr1 in the development of dopa-minergic neurons and Parkinson's disease.” Prog Neurobiol 77(1-2): 128-38.
Lund B W, Knapp A E, Piu F, Gauthier N K, Begtrup M, Hacksell U, Olsson R. (2005) Discovery of a potent, orally available, and isoform-selective retinoic acid beta2 receptor agonist. J Med Chem 48, 7517-7519.
McFarland K, Spalding T A, Hubbard D, Ma J N, Olsson R, Burstein E S. (2013) Low Dose Bexarotene Treatment Rescues Dopamine Neurons and Restores Behavioral Function in Models of Parkinson's Disease. ACS Chem Neurosci. 11:1430-1438.
Mijatovic J., Airavaara M., Planken A., Auvinen P., Raasmaja A., Piepponen T. P., Costantini F., Ahtee L. and Saarma M. (2007) Constitutive Ret activity in knock-in multiple endocrine neoplasia type B mice induces profound elevation of brain dopamine concentration via enhanced synthesis and increases the number of TH positive cells in the substantia nigra. J. Neurosci. 27, 4799-4809.
Ohta K, Kawachi E, Inoue N, Fukasawa H, Hashimoto Y, Itai A, Kagechika H. Retinoidal pyrimidinecarboxylic acids. (2000) Unexpected diaza-substituent effects in retinobenzoic acids. Chem Pharm Bull 48, 1504-1513.
Ordentlich, P., Y. Yan, et al. (2003). “Identification of the antineoplastic agent 6-mercaptopurine as an activator of the orphan nuclear hormone receptor Nurr1.” J Biol Chem 278(27): 24791-9.
Pascual A., Hidalgo-Figueroa M., Piruat J. I., Pintado C. O., Gomez-Diaz R. and Lopez-Barneo J. (2008) Absolute requirement of GDNF for adult catecholaminergic neuron survival. Nat. Neurosci. 11, 755-761.
Sacchetti, P., Dwornik, H., Formstecher, P., Rachez, C. and Lefebvre, P. (2002) Re-quirements for heterodimerization between the orphan nuclear receptor nurr1 and retinoid X receptors. J. Biol. Chem., 277, 35088-35096.
Saijo, K., B. Winner, et al. (2009). “A Nurr1/CoREST pathway in microglia and as-trocytes protects dopaminergic neurons from inflammation-induced death.” Cell 137(1): 47-59.
Schinelli, S., Zuddas, A., Kopin, I. J., Barker, J. L., and di Porzio, U. (1988) 1-Methyl-4-phenyl-1,2,3,6-tetrahydropyridine metabolism and 1-methyl-4-phenylpyridinium uptake in dissociated cell cultures from the embryonic mesencephalon. J. Neurochem. 50, 1900-1907.
Shi, Y. (2007). “Orphan nuclear receptors in drug discovery.” Drug Discov Today 12(11-12): 440-5.
Tomac A., Lindqvist E., Lin L. F., Ogren S. O., Young D., Hoffer B. J. and Olson L. (1995) Protection and repair of the nigrostriatal dopaminergic system by GDNF in vivo. Nature 373, 335-339.
Umemiya H, Fukasawa H, Ebisawa M, Eyrolles L, Kawachi E, Eisenmann G, Gronemeyer H, Hashimoto Y, Shudo K, Kagechika H. (1997) Regulation of retinoidal actions by diazepinylbenzoic acids. Retinoid synergists which activate the RXR-RAR heterodimers. J Med Chem 40, 4222-4234.
Wallen-Mackenzie A, Mata de Urquiza A, Petersson S, Rodriguez F J, Friling S, Wagner J, Ordentlich P, Lengqvist J, Heyman R A, Arenas E, Perlmann T. (2003) Nurr1-RXR heterodimers mediate RXR ligand-induced signaling in neuronal cells. Genes Dev 17, 3036-3047.
Zetterstrom, R. H., L. Solomin, et al. (1997). “Dopamine neuronagenesis in Nurr1-deficient mice.” Science 276(5310): 248-50.

1. A method for treating a neurodegenerative disease or disorder, or stroke using a combination of one or more RXR agonist and/or one or more Nurr1 agonist and one or more trophic factor, or pharmaceutically acceptable salts thereof.

2. The method according to claim 1, wherein the neurodegenerative disease or disorder is selected from the group consisting of Parkinson's disease, Alzheimer's disease, Huntington's disease, frontotemporal lobar degeneration associated with protein TDP-43 (FTLD-TDP, Dementia with Lewy bodies (DLB), vascular dementia, Amyotrophic lateral sclerosis (ALS), Mild Cognitive Impairment (MCI), Parkinson's disease with MCI, and other neurodegenerative related dementias due to changes in the brain caused by ageing, disease or trauma, or spinal cord injury.

3. The method according to claim 1, wherein the RXR agonist is selected from the group consisting of Bexarotene, LGD 100268, LGD 100324, 3,7-dimethyl-6(S),7(S)-methano,7-[1,1,4,4-tetramethyl-1,2,3,4-tetrahydronaphth-7-yl]2(E), 4(E)heptadienoic acid, p[3,5,5,8,8-pentamethyl-1,2,3,4-tetrahydro-2-naphthyl-(2carbonyl)]-benzoic acid, p(5,5,8,8-tetramethyl-,1,2,3,4-tetrahydro-3-isopropyl-2-naphthyl-(2-carbonyl)]-benzoic acid, p[5,5,8,8-tetramethyl-1,2,3,4-tetrahydro-3-isopropyl 2-naphthyl-(2-methano)]-benzoic acid, p[5,5,8,8-tetramethyl-1,2,3,4-tetrahydro-3-ethyl-2-naphthyl-(2-methano)]-benzoic acid, p[(5,5,8,8-tetramethyl-1,2,3,4-tetrahydro-3-bromo-2-naphthyl-(2-methano)]-benzoic acid, p[5,5,8,8-tetramethyl-1,2,3,4-tetrahydro-3]-chloro-2-naphthyl-(2-methano)-benzoic acid, p[3,5,5,8,8-pentamethyl-1,2,3,4-tetrahydro-2-naphthyl-(2methano)]-benzoic acid, p[3,5,5,8,8-pentamethyl-1,2,3,4-tetrahydro-2-naphthyl-(2-hydroxymethyl)]benzoic acid, p[5,5,8,8-tetramethyl-1,2,3,4-tetrahydro-3-bromo-2-naphthyl-(2-carbonyl)]-benzoic acid, p[5,5,8,8-tetramethyl-1,2,3,4-tetrahydro-3-chloro-2-naphthyl-(2-carbonyl)]-benzoic acid, p[5,5,8,8-tetramethyl-1,2,3,4-tetrahydro-3-hydroxy-2-naphthyl-(2-carbonyl)]-benzoic acid, p[5,5,8,8-tetramethyl-1,2,3,4-tetrahydro-3-ethyl-2-naphthyl-(2-carbonyl)]-benzoic acid, p[3,5,5,8,8-pentamethyl-1,2,3,4-tetrahydro-2-naphthyl-(2-thioketo)]-benzoic acid, p[3,5,5,8,8-pentamethyl-1,2,3,4-tetrahydro-2-naphthyl-(2-carbonyl)]-N-(4-hydroxyphenyl)benzamide, p[3,5,5,8,8-pentamethyl-1,2,3,4-tetrahydro-2-naphthyl-(2-methano)]-N-(4-hydroxyphenyl)benzamide, 2-[1-(3,5,5,8,8-pentamethyl-5,6,7,8-tetrahydro-2-naphthyl)ethenyl]pyridine-5-carboxylic acid; ethyl 2-[1-(3,5,5,8,8-pentamethyl-5,6,7,8-tetrahydro-2-naphthyl)ethenyl]pyridine-5-carboxy late; 2-[1-(5,5,8,8-tetramethyl-5,6,7,8-tetrahydro-2-naphthyl) ethenyl]pyridine-5-carboxylic acid; 4-[1-(3,5,5,8,8-pentamethyl-5,6,7,8-tetrahydro-2-naphthyl)epoxy]benzoic acid; 4-[1-(3,5,5,8,8-pentamethyl-5,6,7,8-tetrahydro-2-naphthyl)cyclopropyl]benzoic acid; 4-[1-(3,5,5,8,8-pentamethyl-5,6,7,8-tetrahydro-2-naphthyl)ethenyl]benzenetetrazole; 5-[1-(3,5,5,8,8-pentamethyl-5,6,7,8-tetrahydro-2-naphthyl)ethenyl]pyridine-2-carboxylic acid; 2-[1-(3,5,5,8,8-pentamethyl-5,6,7,8-tetrahydro-2-naphthyl)cyclopropyl]pyridine-5-carboxylic acid; methyl 2-[1-(3,5,5,8,8-pentamethyl-5,6,7,8-tetrahydro-2-naphthyl)cyclopropyl]pyridine-5-carboxylate; 3-methyl-7-propyl-9-(2,6,6-trimethyl-1-cyclohexen-1-yl)-2E,4E,6Z,8E nonatetranoic acid; 3-methyl-7-isopropyl-9-(2,6,6-trimethyl-1-cyclohexen-yl)-2E,4E,6Z,8E nonatetranoic acid; 3-methyl-7-t-butyl-9-(2,6,6-trimethyl-1-cyclohexen-1-yl)-2E, 4E,6Z,8E nonatetranoic acid; 3-methyl-5-{2-12-(2,6,6-trimethylcyclohexen-1-yl)ethenyl-1-cyclohexyl}-2E,4E-pentadienoc acid; (2E,4E)-3-methyl-5-[1-(3,5,5,8,8-pentamethyl-5,6,7,8-tetrahydro-2-naphthyl)cyclopropyl]penta-2,4-dienoic acid; (2E,4E)-3-methyl-6-(1-[2,6,6-trimethyl-1-cyclohexenyl)ethenyl]cyclopropyl)-2,4-hexadienoic acid; (2E,4E,6Z)-7-(5,5,8,8-tetramethyl-5,6,7,8-tetrahydro-2-naphthyl)-3,8-dimethyl-nona-2,4,6-trienoic acid; (2E,4E,6Z)-7-(3,5,5,8,8-pentamethyl-5,6,7,8-tetrahydro-2-naphthyl)-3-methylocta-2,4,6-trienoic acid; 2-[1-(5,5,8,8-tetramethyl-5,6,7,8-tetrahydro-2-naphthyl)cyclopropyl] pyridine-5-carboxylic acid; 4-[(3,5,5,8,8-pentamethyl-5,6,7,8-tetrahydro-2-naphthyl)carbonyl] benzoic acid oxime; 4-[(3,5,5,8,8-pentamethyl-5,6,7,8-tetrahydro-2-naphthyl)carbonyl] benzoic acid methyloxime; 4-[1-(2-methyl-4-t-butylphenyl)ethenyl] benzoic acid; 4-[1-(2-methyl-4-t-butylphenyl)cyclopropyl] benzoic acid; 4-[(2-methyl-4-t-butylphenyl)carbonyl] benzoic acid; 4-[(2-methyl-4-t-butylphenyl)carbonyl] benzoic acid oxime; and 4-[1-(2-methyl-4-t-butylphenyl)carbonyl] benzoic acid methyloxime; or a pharmaceutically acceptable salt thereof.

4. The method according to claim 1, wherein the RXR agonist is bexarotene or 3,7-dimethyl-6(S),7(S)-methano,7-[1,1,4,4-tetramethyl-1,2,3,4-tetrahydronaphth-7-yl]2(E),4(E) heptadienoic acid.

5. The method according claim 1, wherein the trophic factor is selected from the group consisting of glial-cell-line-derived neurotrophic factor (GDNF) or analogs of GDNF, Neurturin (NTN), brain-derived neurotrophic factor (BDNF), fibroblast growth factor (FGF), fibroblast growth factor 9 (FGF-9), ciliary neurotrophic factor (CNTF), bone morphogenetic proteins (BMPs), mesencephalic astrocyte-derived neurotrophic factor (MANF), Cerebral dopamine neurotrophic factor (CDNF), hepatocyte growth factor (HGF), nerve growth factor (NGF), Neurotrophin 3 (NT-3), Neurotrophin 4/5 (NT-4/5), Neurotrophin 6 (NT-6), Neurotrophin 7 (NT-7), artemin (ARTN), and persephin (PSPN), CERE-120 (AAV2 vector encoding human neurturin), granulocyte macrophage colony-stimulating factor (GM-CSF), Insulin-like growth factor (IGF)-1, and transforming growth factor beta 1 (IGF-beta1).

6. The method according to claim 5, wherein the trophic factor is selected from the group consisting of glial-cell-line-derived neurotrophic factor (GDNF), Neurturin (NTN), and brain-derived neurotrophic factor (BDNF).

7. The method according to claim 1, wherein the RXR agonist is bexarotene and the trophic factor is GDNF.

8. The method according to claim 1, wherein the RXR agonist is bexarotene or 3,7-dimethyl-6(S),7(S)-methano,7-[1,1,4,4-tetramethyl-1,2,3,4-tetrahydronaphth-7-yl]2(E),4(E) heptadienoic, and the trophic factor is selected from the group consisting of GDNF, NRTN, IGF-1, NT-4, and or NGF, and wherein the trophic factor is optionally administered by injection of a vector expressing the gene encoding the trophic factor.

9. The method according to claim 1, wherein the Nurr1 agonist is selected from the group consisting of a compound of the formula:

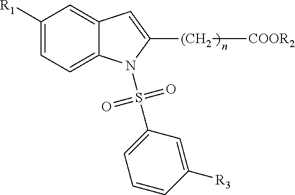

| Ex. | R₁ | n | R₂ | R₃ |
|---|---|---|---|---|
| 1 | 5-CF₃ | 3 | CH₃ | CH(CH₃)₂ |
| 2 | 5-CF₃ | 3 | H | CH(CH₃)₂ |
| 3 | 5-Cl | 3 | CH₃ | CH(CH₃)₂ |
| 4 | 5-Cl | 3 | H | CH(CH₃)₂ |
| 5 | 5-CF₃ | 4 | C(CH₃)₃ | CH(CH₃)₂ |
| 6 | 5-CF₃ | 4 | H | CH(CH₃)₂ |
| 7 | 5-CF₃ | 3 | CH₃ | C(CH₃)₃ |
| 8 | 5-CF₃ | 3 | H | C(CH₃)₃ |
| 9 | 5-CF₃ | 4 | CH₃ | C(CH₃)₃ |
| 10 | 5-CF₃ | 4 | H | C(CH₃)₃ |

(6-Chloroimidazo[1,2-a]pyridin-2-yl)(pyridin-2-yl) methanone; Benzoxazol-2-yl(6-chloroimidazo[1,2-a]pyridin-2-yl)methanone; (6-Chloroimidazo[1,2-a]pyridin-2-yl)(3-fiJryl)methanone; (6-Chloroimidazo[1,2-a]pyridin-2-yl)(thien-2-yl)methanone; (6-Chloroimidazo[1,2-a]pyridin-2-yl)(thien-3-yl)methanone; 1,3-Benzodioxol-5-yl(6-chloroimidazo[1,2-a]pyridin-2-yl)methanone; Benzothiazol-2-yl(6-chloroimidazo[1,2-a]pyridin-2-yl) methanone; (6-Methylimidazo[1,2-a]pyridin-2-yl)(thien-2-yl)methanone; and (5-Methylimidazo[1,2-a]pyridin-2-yl)(thien-2-yl)methanone, (6-Pyridin-2-yl)imidazo[1,2-a]pyridin-2-yl)(thien-2-yl) methanone, or pharmaceutically acceptable salts thereof.

10. A composition comprising one or more RXR agonist and/or one or more Nurr1 agonist and one or more trophic factor, or pharmaceutically acceptable salts thereof.

11. The composition according to claim 10 for treating a neurodegenerative disorder or disease, or stroke.

12. The composition according to claim 11, wherein the neurodegenerative disorder or disease is selected from the group consisting of Parkinson's disease, Alzheimer's disease, Huntington's disease, frontotemporal lobar degeneration associated with protein TDP-43 (FTLD-TDP, Dementia with Lewy bodies (DLB), vascular dementia, Amyotrophic lateral sclerosis (ALS), Mild Cognitive Impairment (MCI), Parkinson's disease with MCI, and other neurodegenerative related dementias due to changes in the brain caused by ageing, disease or trauma, or spinal cord injury.

13. The composition according to claim 10, wherein the RXR agonist is selected from the group consisting of Bexarotene, LGD 100268, LGD 100324, 3,7-dimethyl-6(S),7(S)-methano,7-[1,1,4,4-tetramethyl-1,2,3,4-tetrahydronaphth-7-yl]2(E),4(E) heptadienoic acid, p[3,5,5,8,8-pentamethyl-1,2,3,4-tetrahydro-2-naphthyl-(2carbonyl)]-benzoic acid, p(5,5,8,8-tetramethyl-,1,2,3,4-tetrahydro-3-isopropyl-2-naphthyl-(2-carbonyl)]-benzoic acid, p[5,5,8,8-tetramethyl-1,2,3,4-tetrahydro-3-isopropyl 2-naphthyl-(2-methano)]-benzoic acid, p[5,5,8,8-tetramethyl-1,2,3,4-tetrahydro-3-ethyl-2-naphthyl-(2-methano)]-benzoic acid, p[(5,5,8,8-tetramethyl-1,2,3,4-tetrahydro-3-bromo-2-naphthyl-(2-methano)]-benzoic acid, p[5,5,8,8-tetramethyl-1,2,3,4-tetrahydro-3]-chloro-2-naphthyl-(2-methano)-benzoic acid, p[3,5,5,8,8-pentamethyl-1,2,3,4-tetrahydro-2-naphthyl-(2methano)]-benzoic acid, p[3,5,5,8,8-pentamethyl-1,2,3,4-tetrahydro-2-naphthyl-(2-hydroxymethyl)]benzoic acid, p[5,5,8,8-tetramethyl-1,2,3,4-tetrahydro-3-bromo-2-naphthyl-(2-carbonyl)]-benzoic acid, p[5,5,8,8-tetramethyl-1,2,3,4-tetrahydro-3-chloro-2-naphthyl-(2-carbonyl)]-benzoic acid, p[5,5,8,8-tetramethyl-1,2,3,4-tetrahydro-3-hydroxy-2-naphthyl-(2-carbonyl)]-benzoic acid, p[5,5,8,8-tetramethyl-1,2,3,4-tetrahydro-3-ethyl-2-naphthyl-(2-carbonyl)]-benzoic acid, p[3,5,5,8,8-pentamethyl-1,2,3,4-tetrahydro-2-naphthyl-(2-thioketo)]-benzoic acid, p[3,5,5,8,8-pentamethyl-1,2,3,4-tetrahydro-2-naphthyl-(2-carbonyl)]N-(4-hydroxyphenyl)benzamide, p[3,5,5,8,8-pentamethyl-1,2,3,4-tetrahydro-2-naphthyl-(2-methano)]-N-(4-hydroxyphenyl)benzamide, 2-[1-(3,5,5,8,8-pentamethyl-5,6,7,8-tetrahydro-2-naphthyl)ethenyl]pyridine-5-carboxylic acid; ethyl 2-[1-(3,5,5,8,8-pentamethyl-5,6,7,8-tetrahydro-2-naphthyl)ethenyl]pyridine-5-carboxy late; 2-[1-(5,5,8,8-tetramethyl-5,6,7,8-tetrahydro-2-naphthyl) ethenyl]pyridine-5-carboxylic acid; 4-[1-(3,5,5,8,8-pentamethyl-5,6,7,8-tetrahydro-2-naphthyl)epoxy]benzoic acid; 4-[1-(3,5,5,8,8-pentamethyl-5,6,7,8-tetrahydro-2-naphthyl)cyclopropyl]benzoic acid; 4-[1-(3,5,5,8,8-pentamethyl-5,6,7,8-tetrahydro-2-naphthyl)ethenyl]benzenetetrazole; 5-[1-(3,5,5,8,8-pentamethyl-5,6,7,8-tetrahydro-2-naphthyl)ethenyl]pyridine-2-carboxylic acid; 2-[1(3,5,5,8,8-pentamethyl-5,6,7,8-tetrahydro-2-naphthyl)cyclopropyl]pyridine-5-carboxylic acid; methyl 2-[1-(3,5,5,8,8-pentamethyl-5,6,7,8-tetrahydro-2-naphthyl)cyclopropyl]pyridine-5-carboxylate; 3-methyl-7-propyl-9-(2,6,6-trimethyl-1-cyclohexen-1-yl)-2E,4E,6Z,8E nonatetranoic acid; 3-methyl-7-isopropyl-9-(2,6,6-trimethyl-1-cyclohexen-yl)-2E,4E,6Z,8E nonatetranoic acid; 3-methyl-7-t-butyl-9-(2,6,6-trimethyl-1-cyclohexen-1-yl)-2E,4E,6Z,8E nonatetranoic acid; 3-methyl-5-{2-12-(2,6,6-trimethyl-1-cyclohexen-1-yl)ethenyl-1-cyclohexyl}-2E,4E-pentadienoic acid; (2E,4E)-3-methyl-5-[1-(3,5,5,8,8-pentamethyl-5,6,7,8-tetrahydro-2-naphthyl)cyclopropyl]penta-2,4-dienoic acid; (2E,4E)-3-methyl-6-(1-[2,6,6-trimethyl-1-cyclohexenyl)ethenyl]cyclopropyl)-2,4-hexadienoic acid; (2E,4E,6Z)-7-(5,5,8,8-tetramethyl-5,6,7,8-tetrahydro-2-naphthyl)-3,8-dimethyl-nona-2,4,6-trienoic acid; (2E,4E,6Z)-7-(3,5,5,8,8-pentamethyl-5,6,7,8-tetrahydro-2-naphthyl)-3-methylocta-2,4,6-trienoic acid; 2-[1-(5,5,8,8-tetramethyl-5,6,7,8-tetrahydro-2-naphthyl)cyclopropyl] pyridine-5-carboxylic acid; 4-[(3,5,5,8,8-pentamethyl-5,6,7,8-tetrahydro-2-naphthyl)carbonyl] benzoic acid oxime; 4-[(3,5,5,8,8-pentamethyl-5,6,7,8-tetrahydro-2-naphthyl)carbonyl] benzoic acid methyloxime; 4-[1-(2-methyl-4-t-butylphenyl)ethenyl] benzoic acid; 4-[1-(2-methyl-4-t-butylphenyl)cyclopropyl] benzoic acid; 4-[(2-methyl-4-t-butylphenyl)carbonyl] benzoic acid; 4-[(2-methyl-4-t-butylphenyl)carbonyl] benzoic acid oxime; and 4-[1-(2-methyl-4-t-butylphenyl)carbonyl] benzoic acid methyloxime; or a pharmaceutically acceptable salt thereof.

14. The composition according to claim 10, wherein the RXR agonist is bexarotene or 3,7-dimethyl-6(S),7(S)-methano,7-[1,1,4,4-tetramethyl-1,2,3,4-tetrahydronaphth-7-yl]2(E),4(E) heptadienoic acid.

15. The composition according to claim 10, wherein the trophic factor is selected from the group consisting of glial-cell-line-derived neurotrophic factor (GDNF) or analogs of GDNF, Neurturin (NTN), brain-derived neurotrophic factor (BDNF), fibroblast growth factor (FGF), fibroblast growth factor 9 (FGF-9), ciliary neurotrophic factor (CNTF), bone morphogenetic proteins (BMPs), mesencephalic astrocyte-derived neurotrophic factor (MANF), Cerebral dopamine neurotrophic factor (CDNF), hepatocyte growth factor (HGF), nerve growth factor (NGF), Neurotrophin 3 (NT-3), Neurotrophin 4/5 (NT-4/5), Neurotrophin 6 (NT-6), Neurotrophin 7 (NT-7), artemin (ARTN), and persephin (PSPN), CERE-120 (AAV2 vector encoding human neurturin), granulocyte macrophage colony-stimulating factor (GM-CSF), Insulin-like growth factor (IGF)-1, and transforming growth factor beta 1 (TGF-beta1).

16. The composition according to claim 15, wherein the trophic factor is selected from the group consisting of glial-cell-line-derived neurotrophic factor (GDNF), Neurturin (NTN), and brain-derived neurotrophic factor (BDNF).

17. The composition according to claim 10, wherein the RXR agonist is bexarotene and the trophic factor is GDNF.

18. The composition according to claim 10, wherein the Nurr1 agonist is selected from the group consisting of a compound of the formula:

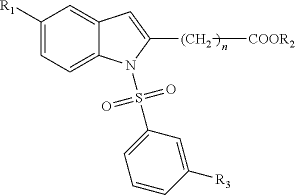

| Ex. | R₁ | n | R₂ | R₃ |
|---|---|---|---|---|
| 1 | 5-CF₃ | 3 | CH₃ | CH(CH₃)₂ |
| 2 | 5-CF₃ | 3 | H | CH(CH₃)₂ |
| 3 | 5-Cl | 3 | CH₃ | CH(CH₃)₂ |
| 4 | 5-Cl | 3 | H | CH(CH₃)₂ |
| 5 | 5-CF₃ | 4 | C(CH₃)₃ | CH(CH₃)₂ |
| 6 | 5-CF₃ | 4 | H | CH(CH₃)₂ |
| 7 | 5-CF₃ | 3 | CH₃ | C(CH₃)₃ |
| 8 | 5-CF₃ | 3 | H | C(CH₃)₃ |
| 9 | 5-CF₃ | 4 | CH₃ | C(CH₃)₃ |
| 10 | 5-CF₃ | 4 | H | C(CH₃)₃ |

(6-Chloroimidazo[1,2-a]pyridin-2-yl)(pyridin-2-yl) methanone; Benzoxazol-2-yl(6-chloroimidazo[1,2-a]pyridin-2-yl)methanone; (6-Chloroimidazo[1,2-a]pyridin-2-yl)(3-fiJryl)methanone; (6-Chloroimidazo[1,2-a]pyridin-2-yl)(thien-2-yl)methanone; (6-Chloroimidazo[1,2-a]pyridin-2-yl)(thien-3-yl)methanone; 1,3-Benzodioxol-5-yl(6-chloroimidazo[1,2-a]pyridin-2-yl)methanone; Benzothiazol-2-yl(6-chloroimidazo[1,2-a]pyridin-2-yl) methanone; (6-Methylimidazo[1,2-a]pyridin-2-yl)(thien-2-yl)methanone; (5-Methylimidazo[1,2-a]pyridin-2-yl)(thien-2-yl)methanone, and (6-Pyridin-2-yl)imidazo[1,2-a]pyridin-2-yl)(thien-2-yl) methanone, or pharmaceutically acceptable salts thereof.
